(19) United States
(12) Patent Application Publication
(10) Pub. No.: US 2007/0244370 A1
(43) Pub. Date: Oct. 18, 2007

(54) MINIATURE WIRELESS APPARATUS FOR COLLECTING PHYSIOLOGICAL SIGNALS OF ANIMALS

(75) Inventors: Terry B. J. KUO, Ji-An Township (TW); Cheryl C. H. Yang, Ji-An Township (TW)

Correspondence Address: EGBERT LAW OFFICES, 412 MAIN STREET, 7TH FLOOR, HOUSTON, TX, 77002, US

(73) Assignees: Terry B. J. KUO, Ji-An Township (TW); Cheryl C. H. Yang, Ji-An Township (TW); ENJOY RESEARCH INC., Ji-An Township (TW)

(21) Appl. No.: 11/696,820

(22) Filed: Apr. 5, 2007

(30) Foreign Application Priority Data
Apr. 12, 2006 (TW) 095112957

(51) Int. Cl. A61B 5/00; A61B 5/04

(52) U.S. Cl. 600/300; 128/903; 600/544; 600/546; 600/549; 600/509

(57) ABSTRACT

A miniature wireless apparatus for collecting physiological signals of animals includes a connector, an amplifier module, a microcontroller, a radio module and a power supply. The connector is attached to an animal to be tested by buckling a joint of the connector to a joint of a connector on the animal to be tested, so as to collect various physiological signals. The amplifier module amplifies each of the physiological signals by an appropriate gain to generate an amplified physiological signal. The microcontroller performs analog-to-digital conversion and data compression on the amplified physiological signal to generate a digital physiological signal. The radio module modulates the digital physiological signal, and then transmits the modulated digital physiological signal to a remote receiver in a wireless transmission manner. The radio module receives remote wireless signals as well. The power supply is configured to provide power to the above circuits.

### CROSS-REFERENCE TO RELATED U.S. APPLICATIONS

Not applicable.

### STATEMENT REGARDING FEDERALLY SPONSORED RESEARCH OR DEVELOPMENT

Not applicable.

### NAMES OF PARTIES TO A JOINT RESEARCH AGREEMENT

Not applicable.

### REFERENCE TO AN APPENDIX SUBMITTED ON COMPACT DISC

Not applicable.

### BACKGROUND OF THE INVENTION

1. Field of the Invention

The present invention relates to an apparatus for collecting physiological signals. More particularly, the present invention relates to a miniature wireless apparatus for collecting physiological signals of animals.

2. Description of Related Art Including Information Disclosed Under 37 CFR 1.97 and 37 CFR 1.98.

Signals such as the electrocardiogram (ECG), electroencephalogram, respiration, and body temperature are indices of life. Indices of sleep and autonomic nervous system functions can be obtained on the basis of analysis of these signals together with electromyogram (EMG) signals. Thus, the collecting and analysis of physiological signals is helpful to the understanding of a broad range of medical information and to future medical applications. It is very important to establish the collecting and analysis mode of physiological signals of rats and mice that are commonly used in animal experiments, as the mode is an outpost of the deep understanding of normal physiological conditions and possible pathological mechanisms of human beings.

FIG. 1 is an ECG signal of the heartbeat. Generally, the peak waveband is referred to as the QRS wave, in which the point from which the waveform first turns upward is referred to as the Q point, the top point is referred to as the R point, and the bottom point is referred to as the S point. In a QRS recognition program, the QRS wave is derived from the micro physiological signals with a peak detection program, and parameters including the amplitude and the duration are measured for each QRS wave. The average value and standard deviation of the parameters are calculated to establish a standard template. Then, each QRS wave is compared with the template.

The heart rate variability (HRV) analysis is a method to analyze the physiological function of the heart on the basis of the heartbeat period sequence. The standard analysis procedure was defined by Task Force of the European Society of Cardiology and the North America Society of Pacing and Electrophysiology in 1996, and was modified by Kuo et al. in 1999. The principle is substantially as follows.
1. First, information about the heartbeat cycle is obtained. Normally, each heartbeat is defined with the R waves in the ECG, and the time difference between an R wave and the next R wave is a heartbeat period RR.
2. The existence of significant fluctuation, for example larger than three times the standard difference, in continuous RR sequences, indicates that heartbeat irregularities or noises may exist. If heartbeat irregularities exist, alert should be provided as the subject's condition might be life-threatening. If noises exist, the technology of measuring and analysis should be improved.
3. If continuous RR sequences do not have large fluctuations, more detailed numerical analysis, such as the spectrum analysis (Kuo et al., 1999) and non-linear analysis (Kuo & Yang, 2002), can be performed on the RR sequences.

In past research of the interaction between sleep and cyclical rhythm, wired systems were applied to study the changes in autonomic nervous systems during the sleep of humans and rats (Yang et al., 2002-2003), and it has been confirmed that the HRV reflecting the regulation of the cardiac autonomic nerve signal has periodic changes according to the sleep stages. Furthermore, it has been found that obvious negative correlation exists between the depth of sleep and the activity of the sympathetic nerves.

The stages of sleep are mainly defined according to the electroencephalogram (EEG), EMG, and electrooculogram. If the sleep stage is easily identified, there is a better possibility of understanding the occurrence and prevention of many sleep-related diseases. The measurement of EEG alone can show the occurrence of many diseases, such as epilepsy and Alzheimer's disease. If the respiration signal can be measured at the same time, diseases related to respiration in sleep such as sleep apnea can be diagnosed. If the heart rate or HRV analysis is performed additionally, the relationship between sleep and hypertension can be understood more thoroughly. The monitoring and analysis of physiological signals in sleep will be indispensable to clinical medicine, and widespread use of the measurement of the signals will be beneficial to the prevention, monitoring and diagnosis of many diseases.

Currently, animal experiments related to physiological information performed in non-narcotic states still mainly use wired systems. To monitor various physiological functions, test animals must carry heavy wires. In addition to the fact that the animals cannot move freely, the measured physiological functions are measured under pressure, and may not represent the true physiological phenomena. Therefore, the measurement results might be inaccurate.

### BRIEF SUMMARY OF THE INVENTION

The present invention discloses a miniature apparatus the size of a button for collecting physiological signals using wireless transmission, so as to facilitate the analysis on the physiological signals in various animal experiments.

The miniature wireless apparatus for collecting physiological signals of animals comprises a connector, an amplifier module, a micro controller, a radio module, and a power supply.

The miniature wireless apparatus for collecting physiological signals of animals is buckled to a connector on the animal to be tested, so as to collect a differential physiological signal. First, noises are filtered from the differential physiological signal with an input stage filter to improve the signal to noise ratio of the signal, and then a differential amplifier differentially amplifies the differential physiological signal to generate an amplified physiological signal. Then, a signal with frequency higher than twice the analog/digital sampling frequency of the microcontroller is filtered from the amplified physiological signal with an output stage filter, so as to facilitate the analog/digital sampling of the microcontroller. The analog-to-digital conversion unit of the microcontroller performs the analog-to-digital conversion on the amplified physiological signal generated by the amplifier module with the appropriate voltage resolution and sampling rate, and then a microprocessing operation unit compresses the data to generate a digital physiological signal. The radio module receives the digital physiological signal generated by the micro controller, and a modulator/demodulator modulates the digital physiological signal into a modulated physiological signal, which is then transmitted to a remote end as a wireless physiological signal by a wireless transceiver. Meanwhile, the radio module also uses the wireless transceiver to receive the wireless signal from the remote end.

The advantages of the present invention are as follows. (1) Because of the use of wireless transmission, the motion of test animals is not limited, and the measured physiological signals are closer to the natural state than those measured by the conventional wired systems. (2) Because of the use of wireless transmission, the animals do not contact any examination instruments. Thus interference caused by the instruments (e.g., the AC power source) is avoided, and the quality of signals is more preferable than that of wired systems. (3) Because of the use of digital wireless transmission, there is less distortion in the signal transmission, which is more preferable than many analog wireless transmission systems. (4) Because of the use of digital wireless signal transmission, various (almost indefinite) transmission channels can be used, which is preferable to conventional analog wireless signal transmission. (5) The apparatus can be further miniaturized as all circuits of the amplifier module, micro controller, radio module, and power supply can be integrated in a single circuit board or even a single chip.

### BRIEF DESCRIPTION OF THE SEVERAL VIEWS OF THE DRAWINGS

FIG. 1 shows a graph illustration of an ECG signal of a heartbeat.

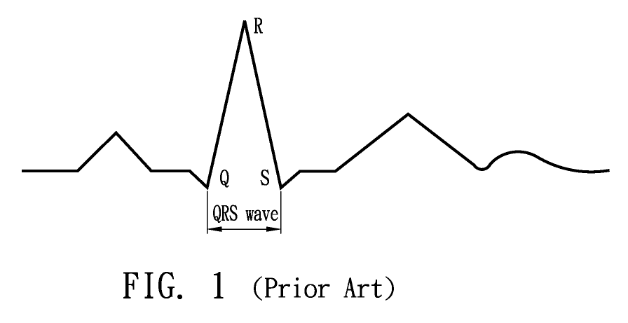

FIG. 2(a) is a schematic view of a block diagram of the wireless system for collecting and analyzing physiological signals of animals according to the present invention.

FIG. 2(b) is a schematic view of a block diagram of the miniature wireless apparatus for collecting physiological signals of animals according to an embodiment of the present invention.

FIGS. 3(a), 3(b), and 3(c) are schematic views of illustrations of structures of the miniature wireless apparatus for collecting physiological signals of animals according to the present invention.

FIG. 3(d) shows a schematic view of a button-type miniature structure of the miniature wireless apparatus for collecting physiological signals of animals according to an embodiment of the present invention.

FIG. 3(e) shows a schematic view of a connector on the bottom surface of the miniature wireless apparatus for collecting physiological signals of animals according to an embodiment of the present invention.

### DETAILED DESCRIPTION OF THE INVENTION

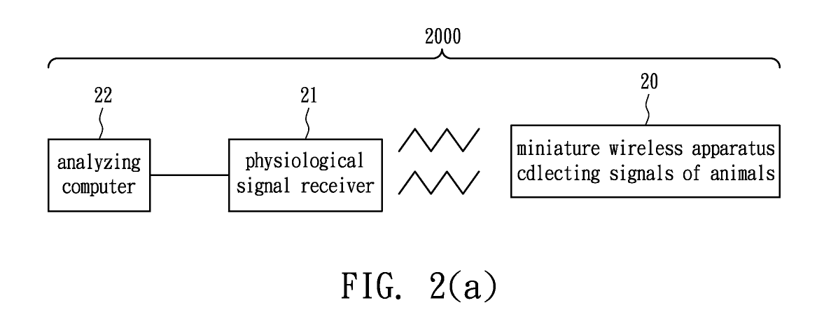

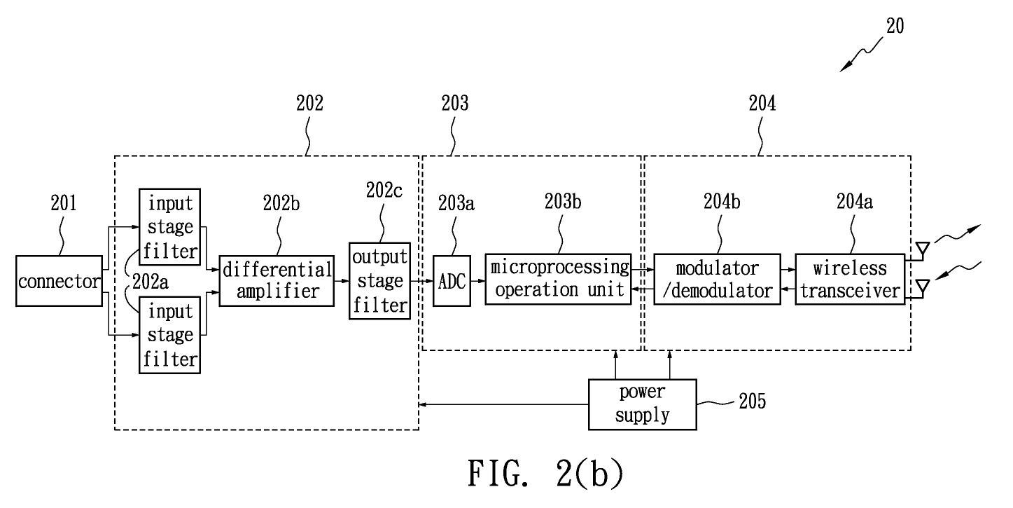

FIG. 2(a) is a schematic view of a block diagram of the wireless system 2000 for collecting and analyzing physiological signals of animals according to the present invention. The system 2000 includes a miniature wireless apparatus 20 for collecting signals of animals, a physiological signal receiver 21, and an analyzing computer 22.

In accordance with an embodiment of the present invention, the miniature wireless apparatus 20 for collecting signals of animals is connected to a rat or a mouse with a connector, so as to collect physiological signals such as EEG, EMG, ECG, respiration and body temperature, and transmit the signals to the remote physiological signal receiver 21. Then the physiological signals are transmitted to the analyzing computer 22 through a computer transmission interface of the physiological signal receiver 21, so as to perform the analysis on the automatic sleep staging and the autonomic nervous function of the heart.

FIG. 2(b) is a schematic view of a block diagram of the miniature wireless apparatus 20 for collecting physiological signals of animals according to an embodiment of the present invention. The apparatus 20 for collecting physiological signals includes a connector 201, an amplifier module 202, a microcontroller 203, a radio module 204, and a power supply 205.

The miniature wireless apparatus for collecting physiological signals of animals 20 is buckled to a joint of a connector on the rat or mouse with a joint of the connector 201, and electrodes are disposed below the connector on the rat or mouse. Thus, mechanical and electrical contacts can be realized at the same time, so as to collect various physiological signals, such as EEG, EMG, ECG, respiration and body temperature of the rat or mouse to be tested.

The amplifier module 202 includes a pair of input stage filters 202a, a differential amplifier 202b, and an output stage filter 202c. After the amplifier module 202 receives a differential physiological signal from the receiver 201, the input stage filters 202a filter out the noise from the signal to increase the signal to noise ratio, and then the differential amplifier 202b performs a differential amplifying to generate an amplified physiological noise. The differential amplifier 202b attenuates the common mode noise, and meanwhile amplifies the differential physiological signal by an appropriate gain, so as to match with the voltage range of the analog-to-digital conversion of the microprocessor 203. Then, the signal with the frequency higher than Nyquist frequency (i.e., twice the sampling frequency in the analog-to-digital conversion of the micro controller) is filtered from the amplified physiological signal by the output stage filter 202c, so as to facilitate the analog-digital sampling of the microcontroller 203. In addition, the impedance of the input end of the amplifier module 202 is larger than 200 kΩ, so as to prevent electric leakage. The input stage filters 202a and the output stage filter 202c can be formed with resistive and capacitive passive elements, and the differential amplifier 202b can be formed with an integrated circuit operational amplifier or instrumentation amplifier.

The microcontroller 203 includes an analog-to-digital conversion unit 203a and a microprocessing operation unit 203b. The analog-to-digital conversion unit 203a performs the analog-to-digital conversion on the amplified physiological signal generated by the amplifier module 202 with the appropriate voltage resolution and sampling rate, and then the microprocessing operation unit 203b compresses the data to generate a digital physiological signal.

The radio module 204 includes a wireless transceiver 204a and a modulator/demodulator 204b. The input end of the radio module 204 connected to the microcontroller 203 forms serial or parallel digital channels, so as to receive the digital physiological signal generated by the microcontroller 203, and the received signal is then modulated to a modulated physiological signal with the carrier of 2.4 GHz by the modulator/demodulator 204b. The modulated physiological signal is then transmitted to the remote physiological signal receiver 21 with the wireless transceiver 204a as a wireless physiological signal. Meanwhile, the wireless transceiver 204a also receives the wireless signal from the remote physiological signal receiver 21. The received signal is demodulated to a digital data signal with the modulator/demodulator 204b, and transmitted to the microcontroller 203 via the digital channel. The wireless signal from the remote physiological signal receiver 21 includes the control signal of the apparatus 20 for collecting physiological signals and the acknowledge signal from the remote physiological signal receiver 21. The acknowledge signal is applied in such a way that, for example, as the digital physiological signal from the microcontroller 203 with its data being compressed is flagged appropriately, and is transmitted to the radio module 204 via the digital channel to transmit and output the wireless physiological signal. By receiving the acknowledge signal transmitted by the remote transceiver, the completeness of the data output of the wireless physiological signal can be guaranteed. The radio module 204 performs the radio transmission and receiving in the 2.4 GHz Industry Science Medical (ISM) frequency band according to international standards.

The power supply 205 can be a miniature battery, a rechargeable miniature battery, or a solar power source, for providing power to all circuits in the apparatus for collecting physiological signals 20.

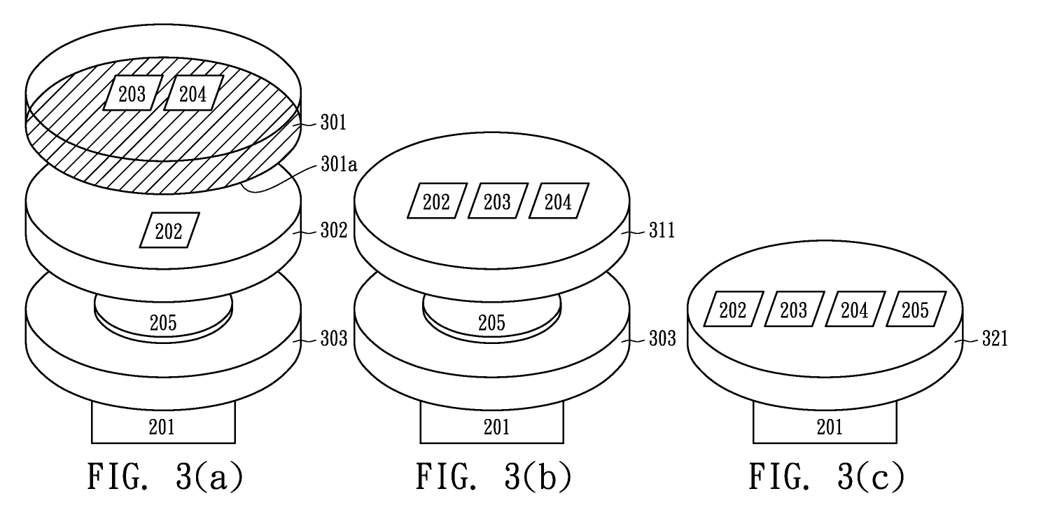

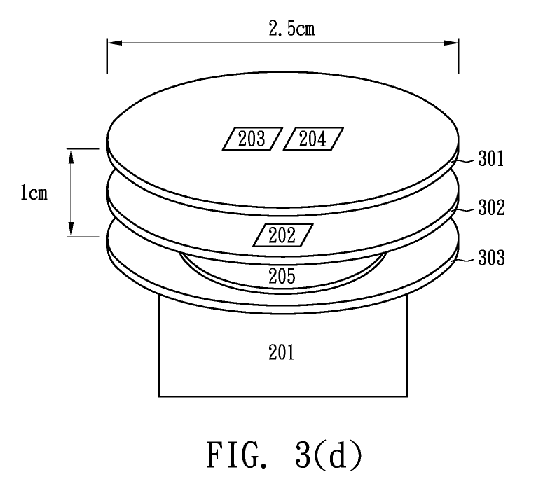

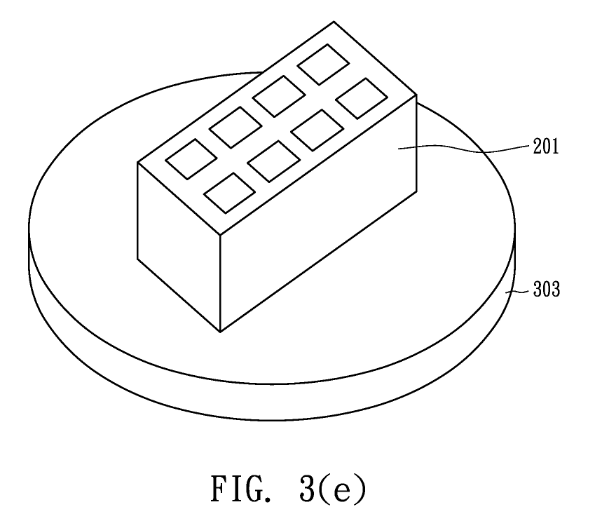

FIGS. 3(a), 3(b), and 3(c) are schematic structural views of the miniature wireless apparatus 20 for collecting physiological signals of animals according to embodiments of the present invention.

FIG. 3(a) shows a multi-layer circuit board structure including circuit board layers 301, 302, and 303. Each of the layers has an upper surface and a bottom surface. The microcontroller 203 and the radio module 204 are disposed on the upper surface of the circuit board layer 301, the amplifier module 202 is disposed on the upper surface of the circuit board layer 302, and the connector 201 is disposed on the bottom surface of the circuit board layer 303. The radio module 204 is disposed on the top circuit board layer 301, which is helpful to the transmission and receiving of wireless signals. As the circuits of the microcontroller 203 and the radio module 204 on the circuit board layer 301 may interfere with the signals from the amplifier module 202 on the circuit board layer 302, a layer of isolation grounding plane 301a is added to the bottom surface of the circuit board layer 301, so as to increase the signal to noise ratio of the analog circuit of the amplifier module 202 on the circuit board layer 302. The power supply 205 can be disposed between the bottom surface of the circuit board layer 302 and the upper surface of the circuit board layer 303, the bottom layer of the circuit board layer 302 can be in direct contact with one of the positive and negative electrodes of the power supply 205 to form a power source layer, and the upper surface of the circuit board layer 303 can be in direct contact with the other electrode of the power supply 205 to form the other power source layer. All circuits excluding the connector 201 shown in FIG. 3(a) can be accommodated in a button-type miniature structure with a diameter of 2.5 cm and a height of 1 cm, as shown in FIG. 3(d). The structure of the connector 201 on the bottom surface of the circuit board layer 303 is as shown in FIG. 3(e).

With improvements in semiconductor technology, the elements 202, 203, and 204 on the circuit board layers 301 and 302 can be integrated on a single circuit board layer 311, and the connector 201 is disposed on the bottom surface of the circuit board layer 303, as shown in FIG. 3(b). In addition, the elements 202, 203, 204, and 205 on the circuit board layers 301, 302, and 303 can be even further integrated on a single circuit board layer 321, as shown in FIG. 3(c). Accordingly, all elements 202, 203, 204, and 205 on the circuit board layer 321 can be integrated into a single chip.

The power supply 205 can be disposed between the circuit board layers 303 and 302, as shown in FIG. 3(a), or disposed between the circuit board layers 303 and 311, as shown in FIG. 3(b), which is helpful for the electrodes on the rat or mouse to be tested to isolate the interference electric waves from the circuits of the microcontroller 203 and the radio module 204. On the other hand, the power supply 205 disposed on a lower layer is helpful to lower the center of gravity of the entire apparatus to improve the stability of the joint between the apparatus for collecting physiological signals 20 and the animal to be tested through a connector.

The above-described embodiments of the present invention are intended to be illustrative only. Numerous alternative embodiments may be devised by those skilled in the art without departing from the scope of the following claims.

1. A miniature wireless apparatus for collecting physiological signals of animals, the apparatus comprising:
a connector means in connection with an animal to be tested for collecting a differential physiological signal;
an amplifier module amplifying the physiological signal to generate an amplified physiological signal;
a microcontroller performing analog-to-digital conversion and data compression on the amplified physiological signal to generate a digital physiological signal;
a radio module modulating the digital physiological signal, transmitting the modulated digital physiological signal to a remote receiver in a wireless transmission manner, and receiving remote wireless signals; and
a power supply providing power to the amplifier module, the microcontroller and the radio module.

2. The miniature wireless apparatus of claim 1, wherein the power supply is a miniature battery or a solar power source.

3. The miniature wireless apparatus of claim 1, wherein the amplifier module comprises a pair of input stage filters connected to the connector, so as to filter noises to improve signal to noise ratio.

4. The miniature wireless apparatus of claim 3, wherein the input stage filters comprise a resistor and a capacitor.

5. The miniature wireless apparatus of claim 1, wherein the amplifier module comprises a differential amplifier for performing a differential amplification on the physiological signal to generate the amplified physiological signal, so as to match with a voltage range of the analog-to-digital conversion of the microcontroller.

6. The miniature wireless apparatus of claim 5, wherein the differential amplifier is formed with an integrated circuit operational amplifier or an instrumentation amplifier.

7. The miniature wireless apparatus of claim 1, wherein the amplifier module comprises an output stage filter for filtering signals with a frequency larger than twice a sampling frequency in the analog-to-digital conversion of the microcontroller, so as to facilitate the analog and digital sampling of the microcontroller.

8. The miniature wireless apparatus of claim 7, wherein the output stage filter comprises a resistor and a capacitor.

9. The miniature wireless apparatus of claim 1, wherein an impedance of an input end of the amplifier module is larger than 200 kΩ, so as to avoid electric leakage.

10. The miniature wireless apparatus of claim 1, wherein the microcontroller comprises:
an analog-to-digital conversion unit connected to the amplifier module for performing analog-to-digital conversion on the amplified physiological signal with a voltage resolution and a sampling rate; and
a microprocessing operation unit connected to the analog-to-digital conversion unit for performing data compression on the digitalized amplified physiological signal to generate the digital physiological signal.

11. The miniature wireless apparatus of claim 1, wherein the radio module comprises:
a modulator/demodulator modulating the digital physiological signal to a modulated physiological signal; and
a wireless transceiver transmitting the modulated physiological signal to the remote receiver in a manner of wireless transmission.

12. The miniature wireless apparatus of claim 1, wherein an input end of the radio module connected to the microcontroller forms serial or parallel digital channels.

13. The miniature wireless apparatus of claim 1, wherein the radio module performs radio transmission and receiving in a 2.4 GHz Industry Science Medical (ISM) frequency band.

14. The miniature wireless apparatus of claim 1, wherein the remote wireless signals comprises a control signal of the wireless apparatus for collecting physiological signals and an acknowledge signal from the remote receiver.

15. The miniature wireless apparatus of claim 1, wherein the physiological signal comprises electroencephalogram (EEG), electromyogram (EMG), electrocardiogram (ECG), respiration, and body temperature.

16. The miniature wireless apparatus of claim 15, wherein the physiological signal is used for automatic sleep staging and cardiac autonomic nervous system function analysis.

17. The miniature wireless apparatus of claim 1, being a multi-layer circuit board structure.

18. The miniature wireless apparatus of claim 17, wherein the microcontroller and the radio module are disposed on a first circuit board layer, wherein the amplifier module is disposed on a second circuit board layer, wherein the connector is disposed on a third circuit board layer, and wherein the second circuit board layer is stacked between the first and the third circuit board layers.

19. The miniature wireless apparatus of claim 18, wherein the microcontroller and the radio module are disposed on an upper surface of the first circuit board layer, wherein the amplifier module is disposed on an upper surface of the second circuit board layer, and wherein the connector is disposed on a bottom surface of the third circuit board layer.

20. The miniature wireless apparatus of claim 18, wherein the power supply is disposed between a bottom surface of the second circuit board layer and an upper surface of the third circuit board layer.

21. The miniature wireless apparatus of claim 18, wherein an isolation grounding plane is formed on a bottom surface of the first circuit board layer, so as to improve a signal to noise ratio of an analog circuit of the amplifier module.

22. The miniature wireless apparatus of claim 18, wherein a bottom surface of the second circuit board layer is in direct contact with one of a positive electrode and a negative electrode of the power supply to form a power source layer, and wherein an upper surface of the third circuit board layer is in direct contact with the other electrode of the power supply to form another power source layer.

23. The miniature wireless apparatus of claim 17, wherein the microcontroller, the radio module, and the amplifier module are disposed on a first circuit board layer, and wherein the connector is disposed on a second circuit board layer.

24. The miniature wireless apparatus of claim 23, wherein the microcontroller, the radio module, and the amplifier module are disposed on an upper surface of the first circuit board layer, and wherein the connector is disposed on a bottom surface of the second circuit board layer.

25. The miniature wireless apparatus of claim 23, wherein the power supply is disposed between a bottom surface of the first circuit board layer and an upper surface of the second circuit board layer.

26. The miniature wireless apparatus of claim 1, wherein the microcontroller, the radio module, the amplifier module and the power supply are disposed on an upper surface of a circuit board layer, and wherein the connector is disposed on a bottom surface of the circuit board layer.

27. The miniature wireless apparatus of claim 1, wherein the microcontroller, the radio module, the amplifier module and the power supply are integrated in a chip.

28. The miniature wireless apparatus of claim 2, wherein the miniature battery is rechargeable.
